(19) United States
(12) Patent Application Publication
(10) Pub. No.: US 2025/0345373 A1
(43) Pub. Date: Nov. 13, 2025

(54) TREATMENT OF INFLAMMATION IN ELDERLY

(71) Applicant: Chr. Hansen A/S, Hoersholm (DK)

(72) Inventors: Sara ENGEL, Hoersholm (DK); Anders DAMHOLT, Hoersholm (DK)

(73) Assignee: Chr. Hansen A/S, Hoersholm (DK)

(21) Appl. No.: 18/869,193

(22) PCT Filed: Jun. 27, 2023
(86) PCT No.: PCT/EP2023/067449

§ 371 Date: Nov. 25, 2024

(30) Foreign Application Priority Data
Jun. 28, 2022 (EP) 22181499.9

(51) Int. Cl. A61K 35/745

(52) U.S. Cl. CPC A61K 35/745

(57) ABSTRACT

The present invention generally relates to human therapeutic effects of Bifidobacterium adolescentis, and particularly alleviating of low-grade inflammation in an elderly population.

### TECHNICAL FIELD

The present invention generally relates to the therapeutic effects of Bifidobacterium adolescentis, and particularly alleviating low-grade inflammation in the elderly.

### TECHNICAL BACKGROUND

Bifidobacteria are natural inhabitants of the gastrointestinal tract possessing genetic adaptations that enable colonization of this harsh and complex habitat. Bifidobacteria interact with key elements of intestinal functioning and contribute to maintaining homeostasis. Recent scientific progress has demonstrated that bifidobacteria, through strain-dependent interactions with the host may reduce mucosal antigen load, improve the intestinal barrier, and induce regulation of local and systemic immune responses.

Due to their recognized benefits to human health bifidobacteria are used as probiotics. Probiotics are “live micro-organisms which, when administered in adequate amounts, confer a health benefit on the host” (FAO/WHO, 2001). About a dozen Bifidobacterium strains with clinically documented effects are commercially available.

Low-grade inflammation is associated with increased risk of Cardiovascular disease (CVD) (Biasucci, 2004; Kaptoge et al., 2010), type 2 diabetes (Barzilay et al., 2001; Dehghan et al., 2007), rheumatoid arthritis (Nielen et al., 2004) and cancer (Taniguchi and Karin, 2018). Low-grade inflammation is defined as slightly increased levels of C-reactive protein (CRP), above 3 mg/L and below 10 mg/L (Imhof et al., 2003; Pearson et al., 2003; Rifai and Ridker, 2003). As an acute-phase protein CRP is a marker of several pathological processes such as infection, cancer, tissue damage, and chronic inflammatory disease (Dinh et al., 2019). Studies have reported associations between aging and increase in C-reactive protein and pro-inflammatory cytokines (Ferrucci and Fabbri, 2018; Franceschi et al., 2017; Li et al., 2011).

In healthy adults B. adolescentis is among the two Bifidobacterium species that predominates in the gut (Matsuki et al., 2004). However the abundance of Bifidobacterium decline with age (Arboleya et al., 2016). Specifically, the abundance of B. adolescentis was shown to decline significantly with age comparing groups of young, middle-aged and older adults ≥60 years (Chen et al., 2021).

Data based on animal and human studies shows that high levels of B. adolescentis in the microbiome is associated with a lower risk of acquiring diseases related to age (Haro et al., 2016; Hevia et al., 2016; Kato et al., 2017; Lim and Kim, 2017; Nobili et al., 2018; Ouwehand et al., 2008). Therefore, it has been suggested that a decline in B. adolescentis may negatively influence inflammation and health.

In healthy adults B. adolescentis is among the two Bifidobacterium species that predominates in the gut (Matsuki et al., 2004). However the abundance of Bifidobacterium decline with age (Arboleya et al., 2016). Specifically, the abundance of B. adolescentis was shown to decline significantly with age comparing groups of young, middle-aged, and older adults ≥60 years (Chen et al., 2021).

### SUMMARY OF THE DISCLOSURE

The present disclosure relates to dietary manipulation of the gut microbiota, preferably the gut microbiota of the elderly, such as supplementation with probiotics, and how this represents a means for preserving a healthy gastrointestinal microbial community and of influencing the inflammatory response in an elderly population. In order to test this, the inventors designed a protocol to study the effect of 12-week supplementation with two doses of B. adolescentis (DSM 29103) vs placebo in an elderly population ≥65 years in a 3-arm parallel designed, placebo-controlled, and randomized study.

According to a first aspect, the disclosure provides a method of alleviating a condition characterized by elevated levels of hs-CRP or TNF-α in a subject, by administering a therapeutically effective amount of Bifidobacterium adolescentis to the subject.

In a preferred embodiment, the B. adolescentis is B. adolescentis DSM 29103.

The condition characterized by elevated levels of hs-CRP or TNF-α may be low-grade inflammation.

The subject may be selected by one or more of the following criteria:
a) age between 65 years and 85 years;
b) hs-CRP level in plasma 3.0-10.0 mg/L; and
c) BMI 18.5-32 kg/m².

The therapeutically effective amount may be 1 billion CFU per day or greater, 10 billion CFU per day or greater.

The therapeutically effective amount may be administered with, for example, 2 capsules per day.

The disclosure provides B. adolescentis for use in alleviating low-grade inflammation.

For example, the B. adolescentis may be B. adolescentis DSM 29103.

The low-grade inflammation may be characterized by hs-CRP level in plasma of 3.0-10.0 mg/L.

For example, the low-grade inflammation is in a subject with age between 65 years and 85 years, and optionally BMI between 18.5-32 kg/m².

The disclosure further provides a composition comprising Bifidobacterium adolescentis and a pharmaceutically acceptable excipient. In one embodiment, the amount viable Bifidobacterium adolescentis is 1 billion CFU or 10 billion CFU.

### BRIEF DESCRIPTION OF THE FIGURES

FIG. 1 is a graphical timeline summarizing the study protocol.

FIG. 2 is a table showing an overview of the biological samples, specimen, schedule of measurements.

FIG. 3 is a table showing the compositions of investigational products.

### DETAILED DESCRIPTION

A bacterial “strain” as used herein refers to a bacterium which remains genetically unchanged when grown or multiplied. The multiplicity of identical bacteria is included.

“Wild type strain” refers to the non-mutated form of a bacterium, as found in nature.

In the present context, the term “derived strain” should be understood as a strain derived from a mother strain by means of e.g. genetic engineering, radiation and/or chemical treatment, and/or selection, adaptation, screening, etc. In specific embodiments the derived strain is a functionally equivalent mutant, e.g. a mutant that has substantially the same, or improved, properties (e.g. regarding probiotic properties) as the mother strain. Such a derived strain is a part of the present invention. The term “derived strain” includes a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant.

A “mutant bacterium” or a “mutant strain” refers to a natural (spontaneous, naturally occurring) mutant bacterium or an induced mutant bacterium comprising one or more mutations in its genome (DNA) which are absent in the wild type DNA. An “induced mutant” is a bacterium where the mutation was induced by human treatment, such as treatment with any conventionally used mutagenization treatment including treatment with chemical mutagens, such as a chemical mutagen selected from (i) a mutagen that associates with or become incorporated into DNA such as a base analogue, e.g. 2-aminopurine or an interchelating agent such as ICR-191, (ii) a mutagen that reacts with the DNA including alkylating agents such as nitrosoguanidine or hydroxylamine, or ethane methyl sulphonate (EMS) or N-methyl-N′-nitro-N-nitroguanidine (NTG), UV- or gamma radiation etc. In contrast, a “spontaneous mutant” or “naturally occurring mutant” has not been mutagenized by man.

A derived strain, such as a mutant, may have been subjected to several mutagenization treatments (a single treatment should be understood one mutagenization step followed by a screening/selection step), but typically no more than 20, no more than 10, or no more than 5, treatments are carried out. In specific embodiments of derived strains, such as mutants, less than 1%, less than 0.1%, less than 0.01%, less than 0.001% or even less than 0.0001% of the nucleotides in the bacterial genome have been changed (such as by replacement, insertion, deletion or a combination thereof) compared to the mother strain.

Mutant bacteria as described above are non-GMO, i.e. not modified by recombinant DNA technology. As an alternative to above preferred method of providing the mutant by random mutagenesis, it is also possible to provide such a mutant by site-directed mutagenesis, e.g. by using appropriately designed PCR techniques or by using a transposable element which is integratable in bacterial replicons.

When the mutant is provided as a spontaneously occurring mutant the above wild-type strain is subjected to the selection step without any preceding mutagenization treatment.

A mutant strain of any of the B. adolescentis strains with accession numbers DSM 29103, DSM 29104, DSM 29106, DSM 29107, DSM 29111, DSM 29102 and DSM 29105 can be obtained by subjecting the strain to mutagenization treatment as described to obtain mutant strains and selecting for mutant strains having the desired properties. Alternatively, a selection is performed for spontaneously occurring mutants.

One embodiment of the disclosure relates to an isolated strain selected from the group consisting of DSM 29103, DSM 29104, DSM 29106, DSM 29107, DSM 29111, DSM 29102 and DSM 29105 and a mutant of any of said deposited strains which is capable of modulating elevated levels of hs-CRP or TNF-α in a subject. A particularly preferred embodiment is B. adolescentis strain with accession number DSM 29103, or a mutant thereof which is capable of modulating elevated levels of hs-CRP or TNF-α in a subject.

By the term “a probiotic product” is meant any product which comprises a probiotic bacterium. A probiotic product comprising a strain according to the invention may be administered in the form of a food product or a dietary supplement. The Bifidobacterium adolescentis may, for example, be incorporated in a dairy product, such as milk, and in particular a fermented dairy product, optionally in combination with other lactic acid bacteria, for example with yogurt ferments, or in other food products such as a snack bar, or beverages such as juice.

The probiotic product comprising Bifidobacterium adolescentis can also be provided as a dietary supplement in the form of a powder, tablet, such as a lozenge or effervescent tablet, pastille, capsule, chewing gum, in individual sachets or as a component of a more general composition such as oil drops, an emulsion or a paste, or in any other suitable carrier determined by those of skill in the art to be an effective carrier for live microorganisms.

Probiotic bacteria are live microorganisms and this can be a challenge during formulation and storage of probiotic products. Probiotic bacteria are especially sensitive towards temperature, moisture content, and oxygen and other ingredients in a formulation matrix. It is preferred that the bacteria of the invention remain viable after prolonged storage in order for the bacteria to impart their beneficial effect upon administration of the probiotic product of the invention to the individual in need thereof.

By the term “viable” is meant that the cell is alive and capable of forming a colony in a petri dish during pour plating or spread plating. The number of viable probiotic bacteria is determined as the number of colony forming units (CFU) by pour plate or spread plate methods with incubation under conditions suitable for growth of the probiotic strain(s). By this method cells capable of growing and forming colonies will be counted. When a number is given in the present specification and claims, it should be understood as CFU/g unless the context indicates otherwise. In some embodiments, the probiotic product of the present invention comprises at least 109 CFU/unit at end of shelf life (EOS). The end of shelf life may be at least 3 months, such as at least 6 months, at least 9 months, at least 12 months, at least 18 months, or at least 24 months.

Using a low water activity ensure a better survival of the probiotic bacteria during storage of the product.

Water activity (aW) is defined as the partial vapor pressure of water in a composition at a specified temperature divided by the standard state partial vapor pressure of water at the same temperature. Water activity thus acts as a measure of the amount of free (i.e. unbound) water in a composition. It may be calculated as: aW=p/p0, where p is the partial vapor pressure of water in the composition and p0 is the vapor pressure of pure water at the same temperature. In probiotic products it is generally preferred that the water activity (aw) is in the range of 0.1-0.2.

The probiotic bacteria to be used in the probiotic products of the invention are generally frozen or freeze-dried. In order to obtain a high viability the bacteria are mixed with a cryoprotectant before they are frozen or freeze-dried.

The term “a cryoprotectant” denotes an excipient that is able to improve the survival during freezing and/or drying and to improve the storage stability of bacteria. The cryoprotectant used herein generally comprises a saccharide.

The saccharide may be a mono-, di-, oligo- or polysaccharide, or a mixture of at least two saccharides. The composition may even comprise three, four or more saccharides.

In some embodiments, the composition comprises a mixture of at least one mono- or disaccharide and at least one oligosaccharide. In other embodiments, the composition comprises a mixture of at least one mono- or disaccharide and at least one polysaccharide.

Monosaccharides useful in the probiotic product of the present disclosure include glucose (also known as dextrose), fructose, ribose and galactose. Disaccharides useful in the probiotic product of the present disclosure include among other sucrose, trehalose, maltose and lactose. The composition may comprise one or more mono- or disaccharides, such as one, two or three or even more different saccharides.

In some embodiments the probiotic product of the disclosure comprises at least one oligosaccharide. An oligosaccharide is a saccharide polymer containing three to nine monosaccharides. Fructo-oligosaccharides (FOS), which are found in many vegetables, consist of short chains of fructose molecules. Galactooligosaccharides (GOS), which also occur naturally, consist of short chains of galactose molecules. These compounds can be only partially digested by humans. The composition may comprise one, two or even more different oligosaccharides.

In some embodiments the probiotic product of the disclosure comprises at least one polysaccharide. Polysaccharides are polymeric carbohydrate molecules composed of more than ten monosaccharide units bound together by glycosidic linkages and on hydrolysis give the constituent monosaccharides or oligosaccharides. They range in structure from linear to highly branched. Examples of polysaccharides to be used in a probiotic product of the disclosure are maltodextrin, cyclodextrin, alginate, pectin, chitosan, starch and inulin. The composition may comprise one, two, three or even more different polysaccharides.

As an example, the cryoprotectant may comprise a mixture of a disaccharide, such as sucrose or glucose, and a polysaccharide, such as maltodextrin.

The addition of oligo- or polysaccharides such as FOS, GOS, inulin and other polysaccharides can assist in reduction of the water activity and has the further advantage that oligo- and polysaccharides are not quite as sweet as mono- and disaccharides and further that they add fibers to the composition.

Polyols (sugar alcohols) have the general formula HOCH2(CHOH)nCH2OH. They are commonly added to foods because of their lower caloric content and less sweetness than sugars. Furthermore they are not broken down by bacteria in the mouth or metabolized to acids, and thus do not contribute to tooth decay,

The composition may further comprise at least one polyol such as erythriol, inositol, isomalt, mannitol, maltitol, sorbitol, or xylitol, or a mixture thereof. Preferred polyols are xylitol, sorbitol and mannitol. The composition may comprise one, two, three or even more different polyols.

The cryoprotectant may further comprise a peptide, protein, protein hydrolysate or a mixture thereof. Examples of peptides and proteins to be used herein are casein, pea, whey, albumin, soy protein, glutamic acid or gelatin, and any isolate or hydrolysate thereof. Other additives, e.g. antioxidants such as ascorbate, sodium citrate, propyl gallate may also be present.

The present disclosure also relates to a probiotic comprising an isolated strain according to the disclosure and a cryoprotectant, such as a saccharide.

Combinations of several species or strains of probiotic bacteria can be used, i.e. 2, 3, 5, 6, 7, 8, 9, 10, 11, 12, 13, 14, 15, 16, 17, 18, 19, 20, 21, 22, 23, 24, 25 or even more of the species and strains listed herein. In presently preferred embodiments, only one, two, three, four or five different strains are present in a probiotic product according to the disclosure.

In addition to the probiotic bacteria, one or more other active ingredients, for example one, two, three, four or more active ingredients selected from the group consisting of vitamins such as vitamin A, D, E, K2, C, B2, B6, B12, biotin, niacin, folic acid; minerals such as zinc, selenium, chromium, copper, calcium, chloride; and vegetable extracts such as cranberry extract/juice, royal jelly could be included in the probiotic product.

It is contemplated that in order to obtain a therapeutical effect, the probiotic product should be administered daily for at least one week, and advantageously for a longer period such as at least 2 weeks, at least 4 weeks, at least 6 weeks, at least 9 weeks, preferably at least 12 weeks, in an amount corresponding to at least 10⁶ CFU, such as at least 10⁷ CFU, preferably at least 10⁸ CFU, generally between 10⁹ CFU and 10¹² CFU of Bifidobacterium adolescentis.

In the present study the probiotic product comprises Bifidobacterium adolescentis as the active ingredient. Bifidobacterium adolescentis may be used as the only active ingredient. Alternatively, the probiotic product as described herein may comprise further compounds of interest such as other bacterial strains, vitamins, prebiotics, fibers or other compounds which may have a beneficial health effect.

The other bacterium may be selected from the group consisting of Bifidobacterium lactis, Lactobacillus rhamnosus, Lactococcus lactis subsp. lactis, Lactococcus lactis subsp. cremoris, Leuconostoc lactis, Leuconostoc mesenteroides subsp. cremoris, Pediococcus pentosaceus, Lactobacillus casei subsp. casei, Streptococcus thermophilus, Bifidobacterium longum, Lactobacillus lactis, Lactobacillus helveticus, Lactobacillus fermentum, Lactobadillus salivarius, Lactobadillus delbrueckii subsp. bulgaricus and Lactobacillus acidophilus.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

TABLE 1
Deposits made at a Depositary institution having acquired the
status of international depositary authority under the Budapest
Treaty on the International Recognition of the Deposit of
Microorganisms for the Purposes of Patent Procedure: Leibniz
Institute DSMZ-German Collection of Microorganisms and Cell
Cultures Inhoffenstr. 7B, 38124 Braunschweig, Germany.

| Strain | Accession No. | Deposit date |
|---|---|---|
| Bifidobacterium adolescentis | DSM 29103 | 2014 Jul. 16 |
| Bifidobacterium adolescentis | DSM 29104 | |
| Bifidobacterium adolescentis | DSM 29106 | |
| Bifidobacterium adolescentis | DSM 29107 | |
| Bifidobacterium adolescentis | DSM 29111 | |
| Bifidobacterium adolescentis | DSM 29102 | |
| Bifidobacterium adolescentis | DSM 29105 | |

### EXAMPLES

### Example 1. Efficacy of B. Adolescentis (DSM 29103) on Level of Markers of Inflammation in an Elderly Population—a Randomized, Double-Blind, Placebo-Controlled, Parallel Group Pilot Study with 12 Weeks Intervention

### Objectives

The primary objective of the study is to evaluate the effectiveness of two doses of B. adolescentis in an elderly population on change in hs-CRP—a marker of low-grade inflammation.

The secondary objectives of the study are to evaluate the effectiveness of two doses of B. adolescentis on:
changes in blood borne biomarkers of systemic inflammation, intestinal barrier function and immune;
changes in intestinal inflammatory biomarkers;
changes in microbiome;
changes in Health-related Quality of Life, mental health and well-being as assessed by the questionnaires short form 36 (SF-36), Depression Anxiety Scale (DASS-21), and WHO (Five) Well Being Index, respectively.

### Investigational Products

Bifidobacterium is a genus of lactic acid- and acetic acid-producing, gram-positive, non-spore forming, non-motile, anaerobic bacteria. They are common constitutes of the microbiota in the human intestinal tract. The investigational product is a high or lower dose of Bifidobacterium adolescentis DSM 29103 in a vegetable capsule. The name of the investigational product is simplified to Bif-038 throughout this document.

The reference product (placebo) is the same capsule but without Bif-038. All products will be produced at Chr. Hansen laboratory in Hoersholm, Denmark, which is certified for food production.

The production batch of Bifidobacterium adolescentis DSM 29103 used in this trial will have a minimum of 0.5*10⁹ or 5*10⁹ CFU per capsule at the time of trial intervention. The daily dose will be 2 capsules in the three arms of the trial. The CFU stability of the investigational product batch will be analysed in parallel with performing this trial and these data will be available and included in the final clinical trial report.

In Europe, strains of Bifidobacterium belonging to the species adolescentis have been granted QPS status by EFSA (EFSA, 2007). This means that the strain used in this trial is considered safe to use in food and as a food supplement.

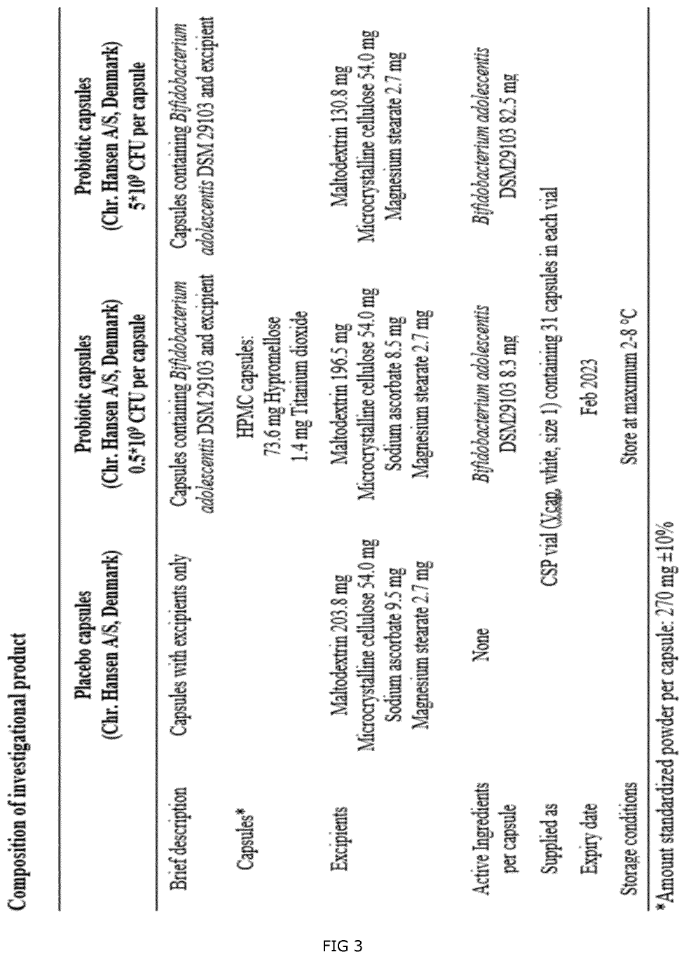

Specific compositions of the investigational products are found in FIG. 3.

The investigational capsules and the placebo capsules are similar in smell, taste, and appearance. All study products will be packaged in identical vials with identical labelling, except for the randomization number.

Study subjects, investigators, study personnel, statisticians and sponsor will be blinded during the entire study until database lock. Only the assigned study supply coordinator at Chr. Hansen A/S will be un-blinded and have access to the randomization list as necessary to perform packaging, labelling and re-supply of study products.

### Methodology

The study is a randomized, double-blind, 3-arm, placebo-controlled pilot study in an elderly population with low-grade inflammation (hs-CRP 3-10 mg/L) comparing two doses daily intake of B. adolescentis at 1 and 10 billion CFU to placebo for 12 weeks.

The subjects will receive either one of the two test products or the placebo product daily for a period of 12 weeks.

During the entire study period, five visits are scheduled. The subjects will participate in two screening visits; a baseline visit, a visit after 6 weeks of intervention and an end-of-study visit after 12 weeks of intervention.

At screening, subjects will be assigned a screening number starting at 1001, according to their chronological entry into the trial. As an example, the first subject will have screening number 1001.

If the subjects are suitable for trial participation, they will receive their randomization number at Visit 2. Randomization numbers will include the stratification number (Female=5; Male=9) and will be allocated sequentially in the order in which the subjects finalize Visit 2.

For example, the first female included will receive the randomization number: 5001, whereas the fifth male included will receive the randomization number: 9005.

The randomization list will be drawn up for N=54 subjects for each strata using the SAS proc plan procedure.

Visit 1 and 2 are screening- and recruitment visits with at least two weeks apart. At the screening visits the eligibility will be checked after the participants have given their written informed consent.

Vaccination status will be obtained as part Medical history.

Repeated CRP measurement are necessary to distinguish between short-term and more sustained CRP elevations.

Only participants with a hs-CRP within 3.0-10.0 mg/L will be invited for the second screening visit.

Participants with repeated hs-CRP levels within 3.0-10.0 mg/L will be invited for baseline visit 3, to be included and randomized in the study.

Before Visit 3, Visit 4 and Visit 5 the subjects will be asked to complete a dietary diary. The subjects will be instructed in how to use the diary. A stool sample kit will be handed out and the subjects will be instructed in how to collect the sample before the baseline visit.

Visit 3 is the baseline visit. Feces samples and blood samples will be collected. Questionnaires will be provided to be filled out at the site. Before the visit the subjects have collected a feces sample and filled in the dietary diary.

Visit 4, after 6 weeks of intervention. Blood samples will be collected. Compliance will be assessed by count of returned unused capsules, and capsules for the remaining 6 weeks of the study will be provided. Questionnaires will be provided to be filled out at the site. A stool sample kit will be handed out and the subjects will be instructed in how to collect the sample before the next visit.

Visit 5 is the end-of-study visit. Feces samples and blood samples will be collected. Questionnaires will be provided to be filled out at the site. Compliance will be assessed by count of returned unused capsules. Before the visit the subjects have collected a feces sample and filled in the dietary diary.

Participants reporting being sick prior to a visit will be rescheduled to a new visit at least 5 days after last symptoms, as CRP levels usually stay elevated for several days after an acute inflammatory condition. In addition, participants should avoid any kind of vaccination minimum±3 days before/after a visit.

At each visit participants will be assessed for Cold or flu-like symptoms with the Jackson questionnaire and have their body temperature measured.

### Visit 1 and 2, Screening Visits:

Blood sample
Questionnaire
Temperature

### Visit 3, Baseline Visit; Day 0:

Blood sample
Feces samples
Questionnaires
3-day food diary
Anthropometrics
Temperature
   Visit 4, after 6 Weeks of Intervention; Week 6:
Blood sample
Compliance
Questionnaires
3-day food diary
Anthropometrics
Temperature

### Visit 5, End-of-Study Visit; Week 12:

Blood sample
Feces samples
Compliance
Questionnaires
3-day food diary
Anthropometrics
Temperature

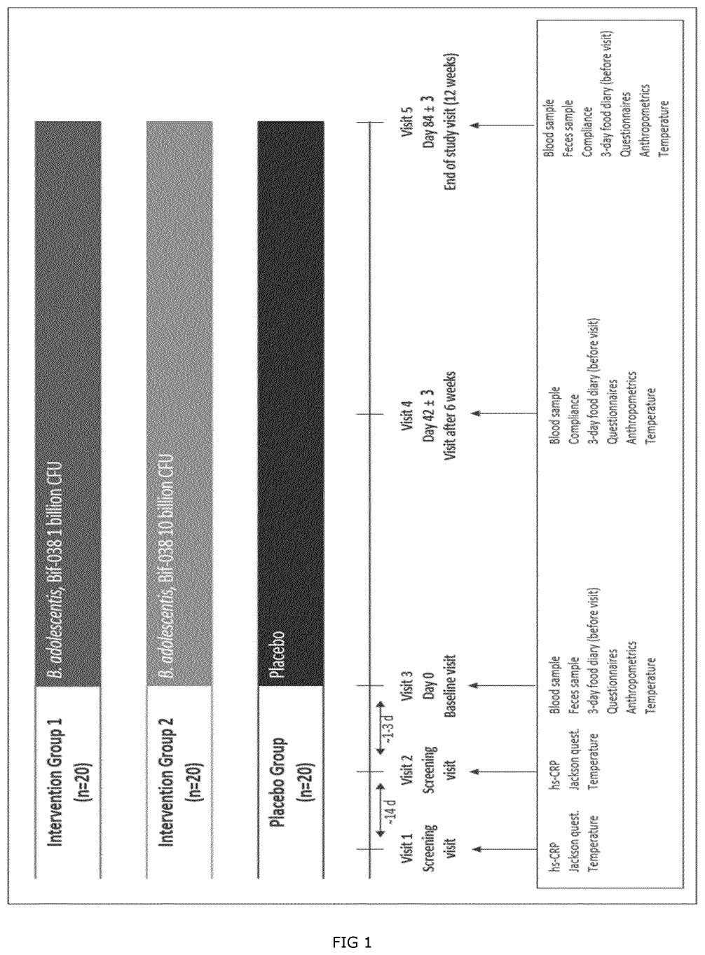

A graphical representation of the study protocol is found in FIG. 1.

### Planned Number of Subjects

No sample size calculations performed due to the exploratory nature of a pilot study design. Planned number to include is 20 subjects per study arm, thus 60 subjects in total. The subjects will be stratified by sex during randomization to ensure equal gender distribution between the study arms. Inclusion of minimum ⅓ of both genders in each arm. Randomized: n=60. Evaluable: n=51 (15% drop-out rate; drop-outs will not be replaced).

### Inclusion Criteria

Willing to participate in the study and comply with its procedures, and able to give written informed consent.

Healthy adults as determined by the investigator:
Age: ≥65 years and s 85 years
hs-CRP: 3.0-10.0 mg/L
BMI: 18.5-32 kg/m²
Controlled, stable hypertension at the discretion of the investigator
Temperature between ≥36.5 and ≤37.3° C.
A stable body weight (≤5% change) over the 3 months prior to screening.

### Test Products

Active arm 1: B. adolescentis, DSM 29103, 1 billion CFU administered orally as 2×0.5B capsules per day.
Active arm 2: B. adolescentis, DSM 29103, 10 billion CFU administered orally as 2×5B capsules per day.
Placebo arm: Placebo capsules administered orally as 2 capsules per day.
Placebo capsules and low dose capsules (0.5B) will be enriched with sodium ascorbate according to levels of high dose capsules (5B).

### Selection of Dosages and Dosage Regimen

The active test product will contain minimum 1*10⁹ CFU and 10*10⁹ CFU of Bif-038 per daily dose in the low and high dose intervention arm, respectively. At these two doses we aim at evaluating if a potential effect of Bif-038 is dose dependent.

### Administration of Study Dose for Each Subject

Subjects will be provided with investigational product at visit 3 (4 vials) and visit 4 (3 vials).

Subjects will return the empty vials and any unused product at visit 4 and visit 5 (end-of-study visit) for assessment of compliance by count of returned capsules.

Subject will receive 168 capsules to be taken over the 12-week period with extra product for 24 days extra in case of loss of product or visit delay.

Subjects will be instructed to consume two capsules per day for 12 weeks with breakfast and preferably just before the breakfast meal (empty stomach).

Study product should be swallowed whole with liquid, but hot liquid or sour liquid should be avoided when swallowing the capsules.

If subjects forget to consume a dose of trial product, it can be taken later the same day, with or without a meal, but an additional dose should not be taken on the following day to compensate for a missing dose on the day before.

Subjects should consume their first dose on the day of visit 3 and consume their last dose on the day before their visit 5.

Subjects should consume their capsule on day of Visit 4 after their study visit. They should not take the capsule when fasting.

In the event of significant worsening, severe or intolerable new GI symptoms, or other AEs after product consumption/use or overdose, the dose may be interrupted after consultation with the sponsor's Medical Monitor representative and PI/Sub-Investigator. The dose may be temporarily interrupted for the shortest amount of time that is possible without increasing the risk to the Subjects continued safety on the trial. No more than 3 days consecutively, if the patient Subject requires dose interruption for more than 3 days with no improvement in GI symptoms and/or other AEs, then consultation with the sponsor representative and PI/Sub-Investigator must occur, and the patient Participant may be withdrawn discontinued from IP and have the option to continue the trial without IP or early participant withdrawal.

### Duration of Intervention

The duration of the trial product/placebo intervention: 12 weeks

### Criteria for Evaluation

Primary endpoint is the effect of daily intake of either 1 billion or 10 billion CFU of B. adolescentis versus placebo on difference in change in hs-CRP from visit 3 (baseline) to visit 5 (end-of-study). Level of hs-CRP will be measured at Visit 1 to Visit 5 as a marker of low-grade inflammation. Subjects hs-CRP must be between 3.0 and 10.0 mg/L at Visit 1 and Visit 2 to be deemed eligible for randomization.

Secondary endpoints are the effect of daily intake of either 1 billion or 10 billion CFU of B. adolescentis versus placebo on difference in change in TNF-α from visit 3 (baseline) to visit 5 (end-of-study).

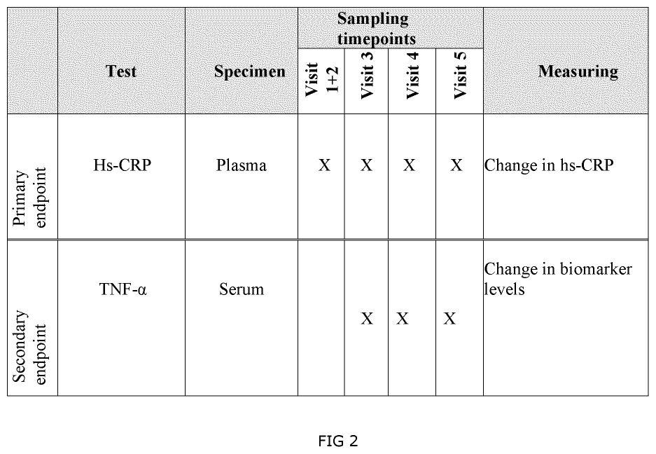

An overview of the biological samples, specimen, schedule of measurements and what to measure is provided in FIG. 2.

### REFERENCES

Andreoletti, O., Lau Baggesen, D., Bolton, D., Butaye, P., Cook, P., Griffin, J., Davies, R., Fernández Escámez, P. S., Griffin, J., Hald, T., Havelaar, A., Koutsoumanis, K., Lindqvist, R., Nguyen-The, C., McLauchlin, J., Nesbakken, T., Prieto, M., Ricci, A., Ru, G., Sanaa, M., Simmons, M., Sofos, J. and Threlfall, J., 2013. Scientific Opinion on the maintenance of the list of QPS biological agents intentionally added to food and feed (2013 update). EFSA Journal John Wiley & Sons, Ltd, 11: 3449.
Arboleya, S., Watkins, C., Stanton, C. and Ross, R. P., 2016. Gut bifidobacteria populations in human health and aging. Frontiers in Microbiology Frontiers Media S. A., 7: 1204.
Barzilay, J. I., Abraham, L., Heckbert, S. R., Cushman, M., Kuller, L. H., Resnick, H. E. and Tracy, R. P., 2001. The relation of markers of inflammation to the development of glucose disorders in the elderly: the Cardiovascular Health Study. Diabetes Diabetes, 50: 2384-2389.
Biasucci, L. M., 2004. CDC/AHA Workshop on Markers of Inflammation and Cardiovascular Disease: Application to Clinical and Public Health Practice: clinical use of inflammatory markers in patients with cardiovascular diseases: a background paper. Circulation Lippincott Williams & Wilkins, 110, doi:10.1161/01.CIR.0000148983.88334.80.
Chen, S., Chen, L., Qi, Y., Xu, J., Ge, Q., Fan, Y., Chen, D., Zhang, Y., Wang, L., Hou, T., Yang, X., Xi, Y., Si, J., Kang, L. and Wang, L., 2021. Bifidobacterium adolescentis regulates catalase activity and host metabolism and improves healthspan and lifespan in multiple species. Nature Aging 2021 1:11 Nature Publishing Group, 1: 991-1001.
Dehghan, A., Kardys, I., De Maat, M. P. M., Uitterlinden, A. G., Sijbrands, E. J. G., Bootsma, A. H., Stijnen, T., Hofman, A., Schram, M. T. and Witteman, J. C. M., 2007. Genetic Variation, C-Reactive Protein Levels, and Incidence of Diabetes. Diabetes American Diabetes Association, 56: 872-878.
Dinh, K. M., Kaspersen, K. A., Mikkelsen, S., Pedersen, O. B., Petersen, M. S., Thørner, L. W., Hjalgrim, H., Rostgaard, K., Ullum, H. and Erikstrup, C., 2019. Low-grade inflammation is negatively associated with physical Health-Related Quality of Life in healthy individuals: Results from The Danish Blood Donor Study (DBDS). PLOS ONE Public Library of Science, 14: e0214468.
EFSA, 2007. Introduction of a Qualified Presumption of Safety (QPS) approach for assessment of selected microorganisms referred to EFSA—Opinion of the Scientific Committee. EFSA Journal Wiley-Blackwell Publishing Ltd, 5, doi:10.2903/J.EFSA.2007.587.
Ferrucci, L. and Fabbri, E., 2018. Inflammageing: chronic inflammation in ageing, cardiovascular disease, and frailty. Nature reviews. Cardiology Nat Rev Cardiol, 15: 505-522.
Franceschi, C., Garagnani, P., Vitale, G., Capri, M. and Salvioli, S., 2017. Inflammaging and “Garb-aging.” Trends in endocrinology and metabolism: TEM Trends Endocrinol Metab, 28: 199-212.
Haro, C., Garcia-Carpintero, S., Alcala-Diaz, J. F., Gomez-Delgado, F., Delgado-Lista, J., Perez-Martinez, P., Rangel ZuRiga, O. A., Quintana-Navarro, G. M., Landa, B. B., Clemente, J. C., Lopez-Miranda, J., Camargo, A. and Perez-Jimenez, F., 2016. The gut microbial community in metabolic syndrome patients is modified by diet. The Journal of nutritional biochemistry J Nutr Biochem, 27: 27-31.
Hevia, A., Milani, C., López, P., Donado, C. D., Cuervo, A., González, S., Suárez, A., Turroni, F., Gueimonde, M., Ventura, M., Sánchez, B. and Margolles, A., 2016. Allergic Patients with Long-Term Asthma Display Low Levels of Bifidobacterium adolescentis. PLoS ONE Public Library of Science, 11, doi:10.1371/JOURNAL.PONE.0147809.
Imhof, A., Fröhlich, M., Loewel, H., Helbecque, N., Woodward, M., Amouyel, P., Lowe, G. D. O. and Koenig, W., 2003. Distributions of C-reactive protein measured by high-sensitivity assays in apparently healthy men and women from different populations in Europe. Clinical chemistry Clin Chem, 49: 669-672.
Kaptoge, S., Di Angelantonio, E., Lowe, G., Pepys, M. B., Thompson, S. G., Collins, R., Danesh, J., Tipping, R. W., Ford, C. E., Pressel, S. L., Walldius, G., Jungner, I., Folsom, A. R., Chambless, L., Ballantyne, C. M., Panagiotakos, D., Pitsavos, C., Chrysohoou, C., Stefanadis, C., Knuiman, M. W., Goldbourt, U., Benderly, M., Tanne, D., Whincup, P., Wannamethee, S. G., Morris, R. W., Kiechl, S., Willeit, J., Mayr, A., Schett, G., Wald, N., Ebrahim, S., Lawlor, D., Yarnell, J., Gallacher, J., Casiglia, E., Tikhonoff, V., Nietert, P. J., Sutherland, S. E., Bachman, D. L., Keil, J. E., Cushman, M., Psaty, B. M., Tracy, R., Tybjerg-Hansen, A., Nordestgaard, B. G., Zacho, J., Frikke-Schmidt, R., Giampaoli, S., Palmieri, L., Panico, S., Vanuzzo, D., Pilotto, L., De La CAmara, A. G., Gómez Gerique, J. A., Simons, L., McCallum, J., Friedlander, Y., Fowkes, F. G. R., Lee, A., Taylor, J., Guralnik, J. M., Phillips, C. L., Wallace, R. B., Blazer, D. G., Khaw, K. T., Brenner, H., Raum, E., Müller, H., Rothenbacher, D., Jansson, J. H., Wennberg, P., Nissinen, A., Donfrancesco, C., Harald, K., Jousilahti, P., Vartiainen, E., Woodward, M., D'Agostino, R. B., Wolf, P. A., Vasan, R. S., Benjamin, E. J., Bladbjerg, E. M., Jorgensen, T., Salomaa, V., Jespersen, J., Dankner, R., Chetrit, A., Lubin, F., Rosengren, A., Wilhelmsen, L., Lappas, G., Eriksson, H., Björkelund, C., Lissner, L., Bengtsson, C., Cremer, P., Nagel, D., Tilvis, R. S., Strandberg, T. E., Kiyohara, Y., Arima, H., Doi, Y., Ninomiya, T., Rodriguez, B., Dekker, J., Nijpels, G., Stehouwer, C. D. A., Rimm, E., Pai, J. K., Sato, S., Iso, H., Kitamura, A., Noda, H., Salonen, J. T., Nyyssönen, K., Tuomainen, T. P., Laukkanen, J. A., Deeg, D. J. H., Bremmer, M. A., Meade, T. W., Cooper, J. A., Hedblad, B., Berglund, G., Engström, G., Verschuren, W. M. M., Blokstra, A., Shea, S., Döring, A., Koenig, W., Meisinger, C., Bueno-De-Mesquita, H. B., Kuller, L. H., Grandits, G., Selmer, R., Tverdal, A., Nystad, W., Gillum, R. F., Mussolino, M., Hankinson, S., Manson, J. E., Knottenbelt, C., Bauer, K. A., Davidson, K., Kirkland, S., Shaffer, J., Korin, M. R., Naito, Y., Holme, I., Nakagawa, H., Miura, K., Ducimetiere, P., Jouven, X., Luc, G., Crespo, C. J., Garcia-Palmieri, M. R., Amouyel, P., Arveiler, D., Evans, A., Ferrieres, J., Schulte, H., Assmann, G., Packard, C. J., Sattar, N., Westendorp, R. G., Buckley, B. M., Cantin, B., Lamarche, B., Desprds, J. P., Dagenais, G. R., Barrett-Connor, E., Wingard, D. L., Bettencourt, R. R., Gudnason, V., Aspelund, T., Sigurdsson, G., Thorsson, B., Trevisan, M., Witteman, J., Kardys, I., Breteler, M. M. B., Hofman, A., Tunstall-Pedoe, H., Tavendale, R., Howard, B. V., Zhang, Y., Best, L., Umans, J., Ben-Shlomo, Y., Davey-Smith, G., Onat, A., Njølstad, I., Mathiesen, E. B., LOchen, M. L., Wilsgaard, T., Ingelsson, E., Basu, S., Cederholm, T., Byberg, L., Gaziano, J. M., Stampfer, M., Ridker, P. M., Ulmer, H., Diem, G., Concin, H., Tosetto, A., Rodeghiero, F., Wassertheil-Smoller, S., Marmot, M., Clarke, R., Fletcher, A., Brunner, E., Shipley, M., Buring, J., Shepherd, J., Cobbe, S., Ford, I., Robertson, M., He, Y., Marin Ibaflez, A., Feskens, E. J. M., Walker, M., Watson, S., Ergou, S., Lewington, S., Pennells, L., Perry, P. L., Ray, K. K., Sarwar, N., Alexander, M., Thompson, A., White, I. R. and Wood, A. M., 2010. C-reactive protein concentration and risk of coronary heart disease, stroke, and mortality: an individual participant meta-analysis. Lancet (London, England) Lancet, 375: 132-140.
Kato, K., Odamaki, T., Mitsuyama, E., Sugahara, H., Xiao, J. zhong and Osawa, R., 2017. Age-Related Changes in the Composition of Gut Bifidobacterium Species. Current Microbiology Springer New York LLC, 74: 987-995.
Khera, A., McGuire, D. K., Murphy, S. A., Stanek, H. G., Das, S. R., Vongpatanasin, W., Wians, F. H., Grundy, S. M. and De Lemos, J. A., 2005. Race and Gender Differences in C-Reactive Protein Levels. Journal of the American College of Cardiology Elsevier, 46: 464-469.
Klein, S. L. and Flanagan, K. L., 2016. Sex differences in immune responses. Nature Reviews Immunology 2016 16:10 Nature Publishing Group, 16: 626-638.
Li, H., Manwani, B. and Leng, S. X., 2011. Aging and Disease Frailty, Inflammation, and Immunity. 466 pp.
Lim, S. M. and Kim, D. H., 2017. Bifidobacterium adolescentis IM38 ameliorates high-fat diet-induced colitis in mice by inhibiting NF-κB activation and lipopolysaccharide production by gut microbiota. Nutrition research (New York, N. Y.) Nutr Res, 41: 86-96.
Matsuki, T., Watanabe, K., Fujimoto, J., Kado, Y., Takada, T., Matsumoto, K. and Tanaka, R., 2004. Quantitative PCR with 16S rRNA-Gene-Targeted Species-Specific Primers for Analysis of Human Intestinal Bifidobacteria. Applied and Environmental Microbiology American Society for Microbiology (ASM), 70: 167.
Monteiro-Junior, R. S., De Tarso Maciel-Pinheiro, P., Da Matta Mello Portugal, E., Da Silva Figueiredo, L. F., Terra, R., Carneiro, L. S. F., Rodrigues, V. D., Nascimento, O. J. M., Deslandes, A. C. and Laks, J., 2018. Effect of Exercise on Inflammatory Profile of Older Persons: Systematic Review and Meta-Analyses. Journal of physical activity & health J Phys Act Health, 15: 64-71.
Neale, E. P., Batterham, M. J. and Tapsell, L. C., 2016. Consumption of a healthy dietary pattern results in significant reductions in C-reactive protein levels in adults: a meta-analysis. Nutrition research (New York, N. Y.) Nutr Res, 36: 391-401.
Nielen, M. M. J., Van Schaardenburg, D., Reesink, H. W., Twisk, J. W. R., Van De Stadt, R. J., Van Der Horst-Bruinsma, I. E., De Gast, T., Habibuw, M. R., Vandenbroucke, J. P. and Dijkmans, B. A. C., 2004. Increased levels of C-reactive protein in serum from blood donors before the onset of rheumatoid arthritis. Arthritis and Rheumatism 50: 2423-2427.
Nobili, V., Putignani, L., Mosca, A., Chierico, F. Del, Vernocchi, P., Alisi, A., Stronati, L., Cucchiara, S., Toscano, M. and Drago, L., 2018. Bifidobacteria and lactobacilli in the gut microbiome of children with non-alcoholic fatty liver disease: which strains act as health players? Archives of Medical Science 14: 81.
Ouwehand, A. C., Bergsma, N., Parhiala, R., Lahtinen, S., Gueimonde, M., Finne-Soveri, H., Strandberg, T., Pitkala, K. and Salminen, S., 2008. Bifidobacterium microbiota and parameters of immune function in elderly subjects. FEMS immunology and medical microbiology FEMS Immunol Med Microbiol, 53: 18-25.
Pearson, T. A., Mensah, G. A., Alexander, R. W., Anderson, J. L., Cannon, R. O., Criqui, M., Fadl, Y. Y., Fortmann, S. P., Hong, Y., Myers, G. L., Rifai, N., Smith, S. C., Taubert, K., Tracy, R. P. and Vinicor, F., 2003. Markers of inflammation and cardiovascular disease: application to clinical and public health practice: A statement for healthcare professionals from the Centers for Disease Control and Prevention and the American Heart Association. Circulation Circulation, 107: 499-511.
Rifai, N. and Ridker, P. M., 2003. Population Distributions of C-reactive Protein in Apparently Healthy Men and Women in the United States: Implication for Clinical Interpretation. Clinical Chemistry Oxford Academic, 49: 666-669.
Taniguchi, K. and Karin, M., 2018. NF-κB, inflammation, immunity and cancer: coming of age. Nature reviews. Immunology Nat Rev Immunol, 18: 309-324.
Valentini, L., Pinto, A., Bourdel-Marchasson, I., Ostan, R., Brigidi, P., Turroni, S., Hrelia, S., Hrelia, P., Bereswill, S., Fischer, A., Leoncini, E., Malaguti, M., Blanc-Bisson, C., Durrieu, J., Spazzafumo, L., Buccolini, F., Pryen, F., Donini, L. M., Franceschi, C. and Lochs, H., 2015. Impact of personalized diet and probiotic supplementation on inflammation, nutritional parameters and intestinal microbiota—The “RISTOMED project”: Randomized controlled trial in healthy older people. Clinical Nutrition Churchill Livingstone, 34: 593-602.

1: A method of alleviating a condition characterized by elevated levels of hs-CRP or TNF-α in a subject, said method comprising administering a therapeutically effective amount of Bifidobacterium adolescentis to the subject.

2: The method according to claim 1, wherein the B. adolescentis is B. adolescentis DSM 29103.

3: The method according to claim 1, wherein the condition characterized by elevated levels of hs-CRP or TNF-α is low-grade inflammation.

4: The method according to claim 1, wherein the subject is selected by at least one of the following criteria:
a) age between 65 years and 85 years (inclusive);
b) hs-CRP level in plasma of 3.0-10.0 mg/L; and
c) BMI of 18.5-32 kg/m².

5: The method according to claim 1, wherein the Bifidobacterium adolescentis is therapeutically effective at an amount of 1 billion CFU per day.

6: The method according to claim 5, wherein the therapeutically effective amount is administered with 2 capsules per day.

7-10. (canceled)

11: A composition comprising Bifidobacterium adolescentis and a pharmaceutically acceptable excipient.

12: The composition according to claim 11, wherein the amount gf viable Bifidobacterium adolescentis is at least 1 billion CFU.

13: The composition according to claim 11, wherein the amount of viable Bifidobacterium adolescentis is at least 10 billion CFU.

14: The composition according to claim 11, wherein the composition is capable of alleviating a condition characterized by elevated levels of hs-CRP or TNF-α in a subject.

15: The composition according to claim 11, wherein the wherein the B. adolescentis is B. adolescentis DSM 29103.

16: The composition according to claim 14, wherein the condition characterized by elevated levels of hs-CRP or TNF-α is low-grade inflammation.

17: The composition according to claim 16, wherein the low-grade inflammation is characterized by hs-CRP level in plasma of 3.0-10.0 mg/L.

18: The method according to claim 1, wherein the Bifidobacterium adolescentis is therapeutically effective at an amount of 10 billion CFU per day.
